(19) United States
(12) Patent Application Publication
(10) Pub. No.: US 2026/0014209 A1
(43) Pub. Date: Jan. 15, 2026

(54) UMBILICAL LINING-DERIVED STEM CELLS FOR TREATING IDIOPATHIC INFLAMMATORY MYOPATHY

(71) Applicant: RESTEM Group, Inc., Corona, CA (US)

(72) Inventors: Rafael Gonzalez, Corona, CA (US); Keith March, Corona, CA (US)

(21) Appl. No.: 19/264,712

(22) Filed: Jul. 9, 2025

Related U.S. Application Data
(60) Provisional application No. 63/669,017, filed on Jul. 9, 2024.

(51) Int. Cl. A61K 35/51; A61P 21/00; A61P 37/06

(52) U.S. Cl. CPC A61K 35/51; A61P 21/00; A61P 37/06

(57) ABSTRACT

A method of treating idiopathic inflammatory myopathy in an individual is described. The method involves administering an effective amount of umbilical lining-derived stem cells to the individual. The method may involve reducing or tapering steroid usage in individual afflicted with idiopathic inflammatory myopathy. The method may involve administering a single dose of the umbilical lining-derived stem cells, or administering multiple doses of the umbilical lining-derived stem cells over a defined period of time.

### CROSS-REFERENCE TO RELATED APPLICATIONS

This Application claims priority to U.S. Provisional Patent Application No. 63/669,017, filed Jul. 9, 2024, entitled “Umbilical Lining-Derived Stem Cells For Treating Idiopathic Inflammatory Myopathy,” which is entirely incorporated by reference for all purposes.

### FIELD

This disclosure relates to umbilical lining-derived stem cells and particularly relates to the use of such cells in treating an idiopathic inflammatory myopathy disorder.

### BACKGROUND

Dermatomyositis (DM) and polymyositis (PM) are representative idiopathic inflammatory myopathy disorders characterized by proximal muscle weakness, and by skin involvement in DM. They have a significant adverse impact on quality of life, and are frequently associated with life-threatening complications such as interstitial lung disease (ILD) (Connors, G. R., et al., Chest, 2010. 138(6): p. 1464-74), neoplasia (Qiang, J. K., et al., J Cutan Med Surg, 2017. 21(2): p. 131-136) and cardiac disease (Zhang, L., et al., Clin Cardiol, 2012. 35(11): p. 686-91).

Patients with DM and PM frequently require years of (or, prolonged) treatment with powerful immunosuppressive drugs such as methotrexate, azathioprine and mycophenolate mofetil, which are associated with significant side-effects, ranging from infertility to neoplasia (Brewer, J. D., et al., Arch Dermatol, 2009. 145(12): p. 1391-6.; Pasternak, B., et al., American Journal of Epidemiology, 2013. 177 (11): p. 1296-1305). Novel therapies with an improved risk/benefit profile are needed for idiopathic inflammatory myopathy disorders, including but not limited to DM and PM.

### SUMMARY

In an aspect, a method of treating idiopathic inflammatory myopathy in an individual is disclosed. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of muscle weakness and autoantibodies, including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells. In embodiments, the administering involves administering a single dose of the umbilical lining-derived stem cells. In other embodiments, the administering involves administering multiple doses of the umbilical lining-derived stem cells over a defined period of time.

In another aspect, a method is disclosed for reducing steroid usage or treatment in an individual afflicted or otherwise diagnosed with idiopathic inflammatory myopathy. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of muscle weakness and autoantibodies, including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the steroid comprises prednisone, prednisolone, methyl prednisone, dexamethasone, betamethasone, or triamcinolone, or derivates thereof. In certain embodiments, the steroid comprises prednisone.

In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells. In embodiments, the administering involves administering a single dose of the umbilical lining-derived stem cells. In other embodiments, the administering involves administering multiple doses of the umbilical lining-derived stem cells over a defined period of time.

In another aspect, a method of tapering steroid usage in an individual afflicted with idiopathic inflammatory myopathy is disclosed. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of muscle weakness and autoantibodies, including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the steroid comprises prednisone, prednisolone, methyl prednisone, dexamethasone, betamethasone, or triamcinolone, or derivates thereof. In certain embodiments, the steroid comprises prednisone.

In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells. In embodiments, the administering involves administering a single dose of the umbilical lining-derived stem cells. In other embodiments, the administering involves administering multiple doses of the umbilical lining-derived stem cells over a defined period of time.

In another aspect, a method of improving a symptom associated with idiopathic inflammatory myopathy in an individual is disclosed. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells.

In certain embodiments, the method further involves reducing or tapering a steroid dosage in the individual.

In embodiments, the steroid comprises prednisone, prednisolone, methyl prednisone, dexamethasone, betamethasone, or triamcinolone, or derivates thereof. In certain embodiments, the steroid comprises prednisone.

In embodiments, the administering involves administering a single dose of the umbilical lining-derived stem cells. In certain embodiments, the administering involves administering multiple doses of the umbilical lining-derived stem cells over a defined period of time.

In certain embodiments, the symptom improvement is measured over at least a 1-month duration. In certain embodiments, the symptom improvement is measured over at least a 2-month duration. In embodiments, the symptom improvement is measured over at least a 3-month duration.

In certain embodiments, the symptom improvement correlates with a decrease in a cytokine level in the individual. In certain embodiments, the symptom improvement correlates with a decrease in TNF-α levels in the individual. In certain embodiments, the symptom improvement correlates with a decrease in IL-12 levels in the individual. In certain embodiments, the symptom improvement correlates with a decrease in IL-12p40 levels in the individual. In certain embodiments, the symptom improvement correlates with a decrease in IL-12p70 levels in the individual. In certain embodiments, the symptom improvement correlates with a decrease in MIP-1α levels in the individual. In certain embodiments, the symptom improvement correlates with a decrease in IL-23 levels in the individual.

In another aspect, an effective amount of umbilical lining-derived stem cells for use in treating an individual diagnosed with idiopathic inflammatory myopathy is disclosed.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In certain embodiments, the effective amount comprises at least about 50 million cells. In certain embodiments, the effective amount comprises at least about 100 million cells. In certain embodiments, the effective amount comprises at least about 200 million cells.

### BRIEF DESCRIPTION OF THE DRAWINGS

FIG. 1 refers to a table containing certain Phase 1 trial results as described herein.

FIG. 2 refers to a graph showing Physician Global Disease Score data against defined periods of time.

FIG. 3 refers to a graph showing Physician Global Disease Score data against defined periods of time.

FIG. 4 refers to a graph showing Manual Muscle Testing (MMT-8) data against defined periods of time.

FIG. 5 refers to a graph showing Health Assessment Questionnaire Score data against defined periods of time.

FIG. 6 refers to a graph showing Average score data of Extramuscular Disease against defined periods of time.

FIG. 7 refers to a graph showing Total Improvement Score data against defined periods of time.

FIG. 8 refers to a graph showing Average Percent Reduction from Baseline Dose data against defined periods of time.

FIG. 9A refers to a graph depicting TNF-α (pf/mL) across various tested conditions for Patient 08.

FIG. 9B refers to a graph depicting TNF-α (pf/mL) across various tested conditions for Patient 09.

FIG. 10 refers to Average score data TIS (using a 1-10 scoring system) against defined periods of time. Average of all patients TIS which is a 1-10 score.

FIGS. 11A-11B refer to graphs illustrating significant elevations of IL-12 in patients with idiopathic inflammatory myopathy (IIM) as compared to normal patients without IIM.

FIG. 12 refers to a graph showing the average of IL-12p40 for IIM patients, showing that ULSCs reduce IL-12p40 in patients with IIM, and reduce the inflammatory response of patients' peripheral blood mononuclear cells (PBMCs) when stimulated with LPS.

FIG. 13 refers to a graph showing the average of IL-12p40 for IIM patients, showing that ULSCs reduce the IL-12p40 inflammatory response of patients' PBMCs when stimulated with inflammatory producing beads.

FIG. 14 refers to a graph showing the average of IL-12p70 for IIM patients, showing that ULSCs reduce the IL-12p70 inflammatory response of patients' PBMCs when stimulated with LPS.

FIG. 15 refers to a graph showing significant elevations of the cytokine macrophage inflammatory protein 1α (MIP-1α) in patients with IIM as compared to healthy controls.

FIG. 16 refers to a graph showing the average of MIP-1α for IIM patients, showing that ULSCs reduce the amount of MIP-1α in patients' PBMCs when stimulated with LPS.

FIG. 17 refers to a graph showing IL-23 levels for PBMC baseline, showing that patients with IIM have significantly higher levels of the cytokine IL-23 as compared to healthy controls.

FIG. 18 refers to a graph showing the average of IL-23 for IIM patients, showing that, in patients with IIM, PBMCs have significantly higher levels of IL-23, and when stimulated with LPS, IL-23 further increases significantly more.

FIG. 19 refers to a graph showing the average of IL-23 for IIM patients, showing that, in patients with IIM, PBMCs have significantly higher levels of IL-23, and when stimulated with immune stimulating beads, IL-23 further increases significantly more.

### DETAILED DESCRIPTION

In an aspect, a method of treating idiopathic inflammatory myopathy in an individual is disclosed. The method involves administering an effective amount of umbilical lining-derived stem cells to the individual in need thereof. In embodiments, the administering involves administering a single dose of the umbilical lining-derived stem cells. In other embodiments, the administering involves administering multiple doses of the umbilical lining-derived stem cells over a defined period of time. The defined period of time may be two sequential days or may be spread out 1-, 2-, 3-, or 4-months apart. In certain embodiments, the defined period of time may exceed 4-months between doses.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the effective amount of the umbilical lining-derived stem cells is less than about 50 million cells. For example, the effective amount may be 45 million cells, 40 million cells, 35 million cells, 30 million cells, 25 million cells, 20 million cells, 15 million cells, 10 million cells, or fewer than 10 million cells. In certain embodiments, the effective amount of the umbilical lining-derived stem cells is at least about 50 million cells. In embodiments, the effective amount is at least 60 million cells, at least 70 million cells, at least 80 million cells, or at least 90 million cells. In embodiments, the effective amount of the umbilical lining-derived stem cells is at least about 100 million cells. In embodiments, the effective amount is at least 110 million cells, at least 120 million cells, at least 130 million cells, at least 140 million cells, at least 150 million cells, at least 160 million cells, at least 170 million cells, at least 180 million cells, or at least 190 million cells. In embodiments, the effective amount of the umbilical lining-derived stem cells is at least about 200 million cells. In embodiments, the effective amount is in excess of 200 million cells.

In another aspect, a method of reducing steroid usage in an individual afflicted with idiopathic inflammatory myopathy is disclosed. The method involves administering an effective amount of umbilical lining-derived stem cells to the individual in need thereof. In embodiments, the administering involves administering a single dose of the umbilical lining-derived stem cells. In other embodiments, the administering involves administering multiple doses of the umbilical lining-derived stem cells. In some embodiments, the administering involves administering multiple doses of the umbilical lining-derived stem cells over a defined period of time.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM). In embodiments, the steroid comprises prednisone, prednisolone, methyl prednisone, dexamethasone, betamethasone, or triamcinolone, or derivates thereof. In certain embodiments, the steroid comprises prednisone. In embodiments, the effective amount of the umbilical lining-derived stem cells is less than about 50 million cells. For example, the effective amount may be 45 million cells, 40 million cells, 35 million cells, 30 million cells, 25 million cells, 20 million cells, 15 million cells, 10 million cells, or fewer than 10 million cells. In embodiments, the effective amount of the umbilical lining-derived stem cells is at least about 50 million cells. In embodiments, the effective amount is at least 60 million cells, at least 70 million cells, at least 80 million cells, or at least 90 million cells. In embodiments, the effective amount of the umbilical lining-derived stem cells is at least about 100 million cells. In embodiments, the effective amount is at least 110 million cells, at least 120 million cells, at least 130 million cells, at least 140 million cells, at least 150 million cells, at least 160 million cells, at least 170 million cells, at least 180 million cells, or at least 190 million cells. In embodiments, the effective amount of the umbilical lining-derived stem cells is at least about 200 million cells. In embodiments, the effective amount is in excess of 200 million cells.

In another aspect, a method of tapering steroid usage in an individual afflicted with idiopathic inflammatory myopathy is disclosed. The method involves administering an effective amount of umbilical lining-derived stem cells to the individual in need thereof. In embodiments, the administering involves administering a single dose of the umbilical lining-derived stem cells. In other embodiments, the administering involves administering multiple doses of the umbilical lining-derived stem cells. In some embodiments, the administering involves administering multiple doses of the umbilical lining-derived stem cells over a defined period of time.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM). In embodiments, the steroid comprises prednisone, prednisolone, methyl prednisone, dexamethasone, betamethasone, or triamcinolone, or derivates thereof. In certain embodiments, the steroid comprises prednisone.

In embodiments, the effective amount of the umbilical lining-derived stem cells is less than about 50 million cells. For example, the effective amount may be 45 million cells, 40 million cells, 35 million cells, 30 million cells, 25 million cells, 20 million cells, 15 million cells, 10 million cells, or fewer than 10 million cells. In embodiments, the effective amount of the umbilical lining-derived stem cells is at least about 50 million cells. In embodiments, the effective amount is at least 60 million cells, at least 70 million cells, at least 80 million cells, or at least 90 million cells. In embodiments, the effective amount of the umbilical lining-derived stem cells is at least about 100 million cells. In embodiments, the effective amount is at least 110 million cells, at least 120 million cells, at least 130 million cells, at least 140 million cells, at least 150 million cells, at least 160 million cells, at least 170 million cells, at least 180 million cells, or at least 190 million cells. In embodiments, the effective amount of the umbilical lining-derived stem cells is at least about 200 million cells. In embodiments, the effective amount is in excess of 200 million cells.

In another aspect, a method of improving a symptom associated with idiopathic inflammatory myopathy in an individual is disclosed. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM). In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells. In certain embodiments, the method further involves reducing a steroid dosage in the individual. In embodiments, the steroid comprises prednisone, prednisolone, methyl prednisone, dexamethasone, betamethasone, or triamcinolone, or derivates thereof. In certain embodiments, the steroid comprises prednisone. In embodiments, the administering involves administering a single dose of the umbilical lining-derived stem cells. In other embodiments, the administering involves administering multiple doses of the umbilical lining-derived stem cells. In some embodiments, the administering involves administering multiple doses of the umbilical lining-derived stem cells over a defined period of time. In certain embodiments, the symptom improvement is measured over at least a 1-month duration. In certain embodiments, the symptom improvement is measured over at least a 2-month duration. In embodiments, the symptom improvement is measured over at least a 3-month duration. In embodiments, the symptom improvement is measured over a period extending beyond 3-months. In certain embodiments, the symptom improvement correlates with a decrease in TNF-α levels in the individual. In certain embodiments, the symptom improvement correlates with a decrease in a cytokine level in the individual. In certain embodiments, the cytokine comprises IL-12. In certain embodiments, the cytokine comprises IL-12p40. In certain embodiments, the cytokine comprises IL-12p70. In certain embodiments, the cytokine comprises MIP-1α. In certain embodiments, the cytokine comprises IL-23.

In another aspect, an effective amount of umbilical lining-derived stem cells for use in treating an individual diagnosed with idiopathic inflammatory myopathy is disclosed. In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM). In embodiments, the effective amount of the umbilical lining-derived stem cells is less than about 50 million cells. For example, the effective amount may be 45 million cells, 40 million cells, 35 million cells, 30 million cells, 25 million cells, 20 million cells, 15 million cells, 10 million cells, or fewer than 10 million cells. In certain embodiments, the effective amount of the umbilical lining-derived stem cells is at least about 50 million cells. In embodiments, the effective amount is at least 60 million cells, at least 70 million cells, at least 80 million cells, or at least 90 million cells. In embodiments, the effective amount of the umbilical lining-derived stem cells is at least about 100 million cells. In embodiments, the effective amount is at least 110 million cells, at least 120 million cells, at least 130 million cells, at least 140 million cells, at least 150 million cells, at least 160 million cells, at least 170 million cells, at least 180 million cells, or at least 190 million cells. In embodiments, the effective amount of the umbilical lining-derived stem cells is at least about 200 million cells. In embodiments, the effective amount is in excess of 200 million cells.

Mesenchymal stem/stromal cells (MSCs) are self-renewing, multi-potent stromal cells which act as modulators of immune responses: they have been shown to inhibit dendritic cell (DC) maturation (Spaggiari, G. M., et al., Blood, 2009. 113(26): p. 6576-83) and their ability to present antigen to T cells (Chiesa, S., et al., Proc Natl Acad Sci USA, 2011. 108(42): p. 17384-9); to block B-cell differentiation into plasma cells (Franquesa, M., et al., Stem Cells, 2015. 33(3): p. 880-91) and antibody production (Asari, S., et al., Exp Hematol, 2009. 37 (5): p. 604-15.); as well as to reduce Th17 and Th1 in favor of T regulatory cell responses (Ghannam, S., et al., The Journal of Immunology, 2010. 185(1): p. 302-312).

These MSC activities do not generally require cell-cell contact, and are mediated by multiple secreted factors, including PGE2 (Spaggiari et al., 2009), TGF-β (English, K., et al., Clin Exp Immunol, 2009. 156(1): p. 149-60) and IL-10 (Lee, R. H., et al., Proc Natl Acad Sci USA, 2014. 111(47): p. 16766-71). As a consequence of these pleiotropic activities, MSCs have been shown to possess potent anti-inflammatory effects in animal models of auto-immune disease, including models of colitis (Gonzalez, M. A., et al., Gastroenterology, 2009. 136(3): p. 978-89), graft-versus-host disease (Yanez, R., et al., Stem Cells, 2006. 24(11): p. 2582-91), and rheumatoid arthritis (Gonzalez-Rey, E., et al., Ann Rheum Dis, 2010. 69(1): p. 241-8). In addition, MSCs are poorly immunogenic, as they lack costimulatory CD80 and poorly express human leukocyte antigen (HLA) class II surface antigens (See, F., et al., J Cell Mol Med, 2011. 15(10): p. 2117-29).

MSCs can be obtained through bone marrow aspiration, liposuction, or from the umbilical cord. The last, termed umbilical cord-derived mesenchymal stromal cells (U-MSC), present multiple advantages: they are obtained from a medical waste material, which does not raise ethical concerns; their origin may be associated with a younger cellular phenotype; and studies indicate minimal expression of HLA-DR after activation, suggesting they have a reduced immunogenic potential (Kim, J. H., et al., Stem Cells Int, 2018. 2018: p. 8429042).

Non-randomized, single arm clinical trials employing intravenous administration of MSCs derived from bone-marrow or umbilical cord have demonstrated an excellent safety profile for cellular therapy in drug-resistant systemic lupus erythematosus (81 patients followed for ≥5 years) (Wang, D., et al., Stem Cell Reports, 2018. 10(3): p. 933-941), drug-resistant dermatomyositis or polymyositis (15 patients followed for ≥12 months) (Wang, D., et al., Ann Rheum Dis, 2011. 70(7): p. 1285-8) and in idiopathic pulmonary fibrosis (8 patients followed for 6 months) (Chambers, D. C., et al., Respirology, 2014. 19(7): p. 1013-8). Although most of these studies reported signs of therapeutic efficacy, placebo-controlled studies are required to tease out the benefit of MSC therapy from that of standard, optimized medical therapy.

DM and PM have a complex pathophysiology that may be efficiently counteracted by MSC therapy at various levels. In affected muscles, micro-vascular injury is the first pathological change, preceding perimysial muscle fiber atrophy (Hornung, T. and J. Wenzel, Drugs, 2014. 74(9): p. 981-98). Histological analysis of biopsy samples from affected muscles in DM and PM patients show evidence of endothelial cell sloughing and consequent deposition of membrane attack complexes (Hornung et al., 2014; Lahoria, R., D. Selcen, and A. G. Engel, Brain, 2016. 139(Pt 7): p. 1891-903). MSCs may effectively reduce this microvascular injury pattern, as they have been shown to rescue endothelial cell function in multiple disease scenarios (Feng, Y., et al., 2019. 28(10): p. 674-682; Premer, C., et al., EBioMedicine, 2015. 2(5): p. 467-75); they also promote angiogenesis by producing growth factors such as VEGF (Feng et al., 2019), HGF (Kwon, H. M., et al., Vascul Pharmacol, 2014. 63(1): p. 19-28), or MCP-1 (Kwon et al., 2014), which may counteract the microvascular rarefaction seen in DM/PM. An abnormally up-regulated type-I interferon (IFN) signature is a hallmark of DM and PM (Hornung et al., 2014); plasmacytoid DCs (pDCs) are an important source of type-I interferons (Wenzel, J., et al., Clin Exp Dermatol, 2006. 31(4): p. 576-82), and infiltrating pDCs are abundant in biopsies from diseased muscle (Greenberg, S. A., et al., Ann Neurol, 2005. 57(5): p. 664-78). MSCs may reduce pDC type I IFN production in DM/PM by virtue of their capacity to inhibit DC differentiation, maturation and activation (Spaggiari et al., 2009; Chiesa et al., 2011). MSCs may also reduce the production of autoantibodies, which act as stimulatory factors for pDC IFN production. In this regard, MSC have been shown to inhibit B cell maturation into plasmacytoid (Spaggiari et al., 2009) cells, as well as auto-antibody production. Additionally, they may reduce auto-antibody production by enhancing T regulatory cells development (English et al., 2009; Carrion, F., et al., Lupus, 2010. 19(3): p. 317-22; Luz-Crawford, P., et al., Mesenchymal stem cells generate a CD4+CD25+Foxp3+ regulatory T cell population during the differentiation process of Th1 and Th17 cells. Stem Cell Res Ther, 2013. 4(3): p. 65), which block the activation of allo-reactive Th1 cells (Carneiro, J., et al., Immunol Rev, 2007. 216: p. 48-68; Leon, K., et al., Modelling T-cell-mediated suppression dependent on interactions in multicellular conjugates. J Theor Biol, 2000. 207(2): p. 231-54). Other dysfunctional T cell responses noted in DM/PM may also be effectively modulated by MSC. CD28 cells are increased in the peripheral blood of DM/PM patients, represent the majority of infiltrating CD4+ and CD8+ T cells infiltrating muscle (Fasth, A. E., et al., J Immunol, 2009. 183(7): p. 4792-9), and persist in muscle after glucocorticoid treatment (Pandya, J. M., et al., Arthritis Res Ther, 2016. 18: p. 80). They are resistant to apoptosis, and have augmented cytotoxicity and cytokine-production capabilities (Venalis, P. and I. E. Lundberg, Rheumatology (Oxford), 2014. 53(3): p. 397-405). MSCs have been shown to block allo-reactive CD8+CD28 T cells obtained from human recipients of kidney transplant IL-17 production, which is mainly orchestrated by Th17 cells, is increased in DM/PM affected muscles, and this may be efficiently reduced by MSC-induced downregulation of Th17 cells (Venalis et al., 2014).

### Total Improvement Score (TIS)

Prior to initial enrollment at the beginning of the trial a meeting is held by an appropriate trainer that understands patient assessments, including the MDAAT and other tools described below, in order to optimize homogeneity of assessments throughout the protocol.

When assessing patient scores for physician-led tools, physicians are instructed to consider the prior results as anchors to ensure greater longitudinal consistency. Additionally, the same physician should, as much as feasible, fill out the same assessments at each site. All endpoint-based assessment timepoints are required to be completed by the same investigator who completed the baseline assessment; if this cannot be done it will be considered a protocol deviation. Secondary efficacy-related endpoints include the absolute value as well as the change from baseline at each post-treatment study visit listed in the Schedule in:
Core set measures that comprise the 2016 ACR/EULAR Myositis Response Criteria for Minimal, Moderate, and Major Clinical Response in Adult Dermatomyositis and Polymyositis:
   Physician global disease activity (VAS)
   Patient global disease activity (VAS)
   Manual muscle strength test (MMT)
   Health assessment questionnaire disability index (HAQ-DI)
   Extramuscular disease activity (VAS)
Myositis Disease Activity Assessment Tool (MDAAT)
Steroids
3D-TTE with speckle tracking and strain if available
Spirometry
FI-3

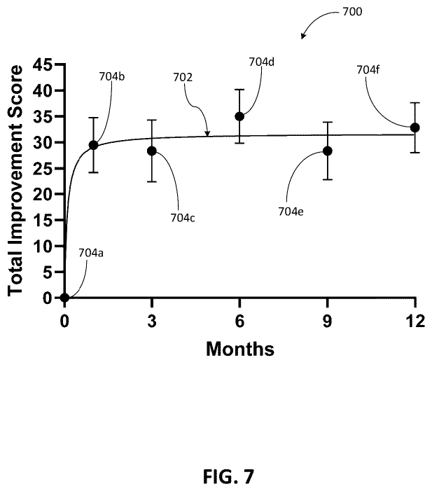

The TIS is not a separate assessment from the previously described Core Set Measures, but is automatically calculated as a composite of these measures, with the addition of muscle enzyme labs, using a web-based tool (see FIG. 7 herein). The results are computed as a change between timepoints where the improvement compared to a given initial timepoint is described as a percentage, with a conventional agreement that a minimally significant TIS is ≥20%. Using this approach, 100% represents complete improvement and 0% would represent no improvement, as a continuous variable.

We will assess disease activity using the Total Improvement Score (TIS) developed by the International Myositis Assessment & Clinical Studies Group, the American College of Rheumatology and the European League against rheumatism. The primary efficacy end-point will measure the magnitude of change in TIS before IV infusion of U-MSC and 6 months after, within each dosing group. The TIS is calculated on a scale from 0 to 100, using the “The Final 2016 ACR/EULAR Criteria for Minimal, Moderate, and Major Clinical Response in Adult Dermatomyositis and Polymyositis” as shown in Table 1:

TABLE 1
The Final 2016 ACR/EULAR Criteria for Minimal, Moderate, and Major
Clinical Response in Adult Dermatomyositis and Polymyositis

| | | Improvement |
|---|---|---|
| | Level of absolute % change in core set | score for |
| Core set measures | measures (improvement) | each level |
| Physician Global | Worsening to ≤5% improvement | 0 |
| Disease Activity | >5% up to ≤15% | 7.5 |
| | >15% up to ≤25% | 15 |
| | >25% up to ≤40% | 17.5 |
| | >40% | 20 |
| Patient Global | Worsening to ≤5% improvement | 0 |
| Disease Activity | >5% up to ≤15% | 2.5 |
| | >15% up to ≤25% | 5 |
| | >25% up to ≤40% | 7.5 |
| | >40% | 10 |
| Manual muscle | Worsening or no change (−ve change | 0 |
| strength test | to ≤5%) | |
| (MMT) | >5% up to ≤15% | 10 |
| | >15% up to ≤25% | 20 |
| | >25% up to ≤40% | 27.5 |
| | >40% | 32.5 |
| Health assessment | Worsening to ≤5% improvement | 0 |
| questionnaire | >5% up to ≤15% | 5 |
| disability index | >15% up to ≤25% | 7.5 |
| | >25% up to ≤40% | 7.5 |
| | >40% | 10 |
| Muscle enzyme | Worsening to ≤5% improvement | 0 |
| (most | >5% up to ≤15% | 2.5 |
| abnormal) | >15% up to ≤25% | 5 |
| | >25% up to <40% | 7.5 |
| | >40% | 7.5 |
| Extramuscular | Worsening to ≤5% improvement | 0 |
| disease activity | >5% up to ≤15% | 7.5 |
| | >15% up to ≤25% | 12.5 |
| | >25% up to ≤40% | 15 |
| | >40% | 20 |

Total Improvement Score (Scale 0-100)

### Physician Global Activity VAS Results

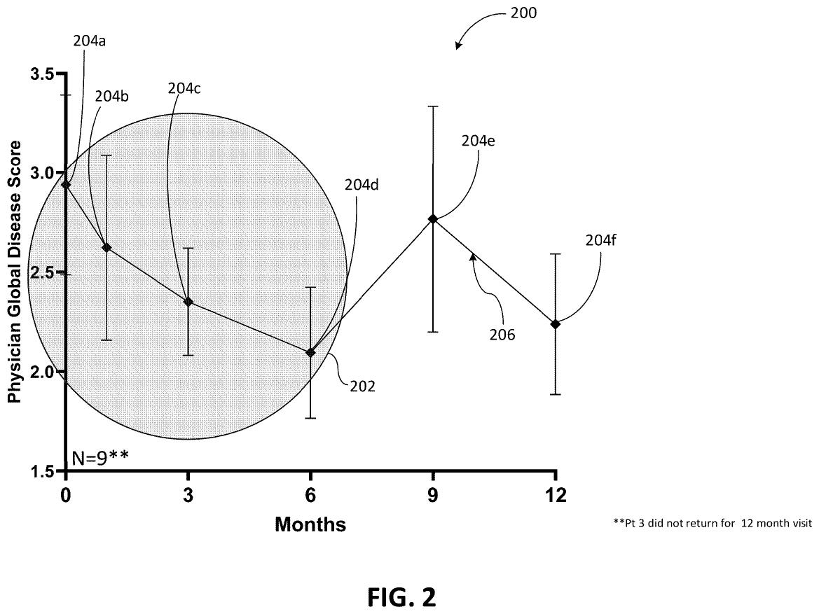

FIG. 2 provides longitudinal results of the Physician Global Activity (PGA) VAS scale for all 9 participants, regardless of dose level. The results suggest that symptomatic pain relief is identified in all dose cohorts through Month 6. The average baseline PGA score across all participants was 2.94(+) 0.45(mean(+)SEM), which by Month 6 had changed to 2.09(+) 0.33. The PGA VAS scale extends from 0.0-10.0 cm, and is generally considered to provide clinically meaningful change with an improvement of ≥20% [45].

### MMT8 Scores

Manual Muscle Testing (MMT8) is a score evaluating the strength of a set of 8 designated muscles tested unilaterally, with a score range of 0-80. In this Phase 1 IIM study, participants demonstrated an overall improvement between baseline and Month 6 of nearly 10 points on the scale, with no SEM overlap: the values were 59.33+3.92 at baseline and 68.11±3.85 at Month 6 (mean±SEM).

The HAQ Disability Index (HAQ-DI) indicates the extent of the respondent's functional ability and is scored with the overall average range between 0 (no disability) to 3 (completely disabled). In the Phase 1 IIM study, participants demonstrated an overall change between baseline and Month 6 of-0.4 on the scale: these were 1.7 at baseline and 1.3 at Month 6. The Minimal Clinical Important Difference (MCID) for this scale has been variably reported as between 0.22 and 0.10. Accordingly, the average change from baseline in this trial was clinically significant throughout the first 6 months.

The TIS is a composite outcome derived from the six core set measures. The results are computed as a change between timepoints where the improvement compared to a given initial timepoint is described as a percentage, with a conventional agreement that a minimally significant TIS is ≥20%. Between baseline and one month following infusion of ULSC, 6 of the 9 participants manifested clinically significant improvement in disease based on the TIS score. Notably, these improvements were observed in the context of reduced prednisone dosage described above.

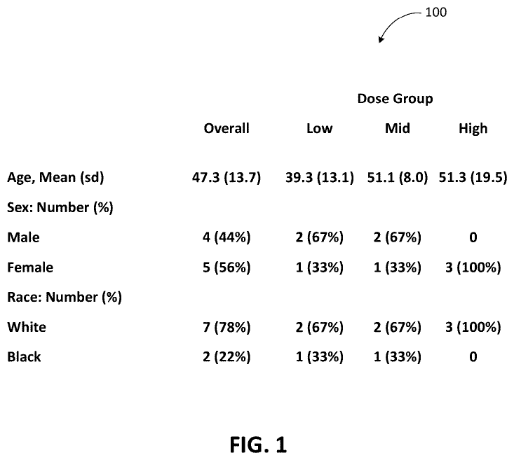

FIG. 1 at 100 provides a summary description of patients with PM/DM involved in the data capture. The average age of all participants in the study was 47.3 years of age with 56% being female and 44% being male. Additionally, 78% of participants were white, and 22% of participants were black. Three cohorts of participants are identified who received a low, mid, or high dose of ULSCs containing 50, 100, and 200 million cells respectively.

FIG. 2 at 200 provides an illustration of the Physician Global Disease Score in participants who were administered ULSCs. Elements 204a through 204d demonstrate an improvement from baseline to 6 months in participants with PM/DM. The Physician Global Disease Score is commonly used in autoimmune diseases that due to their prevalence in the Visual Analog Scale which is psychometric response scale that rates disease-related symptom severity on a scale of 1-10, with 10 being the worst. The assessment is conducted by a physician based on 42 scenarios based on patient synopsis. For example, patients are asked “Considering all the ways your disease has affected you in the last week, how active do you feel your disease is?”; or “Considering all the ways your rheumatic disease/myositis has affected you, how do you feel your rheumatic disease/myositis is today?” or in the “past week?”; or “How have you been feeling in general this past week, in relation to your rheumatic disease/myositis?”. Physician Global Activity (PGA) was measured on a visual analogue scale (VAS) ranging from 0 to 100 mm where higher scores represent worse subjective health. Elements 204a through 204f illustrate the varying score over a 12-month period.

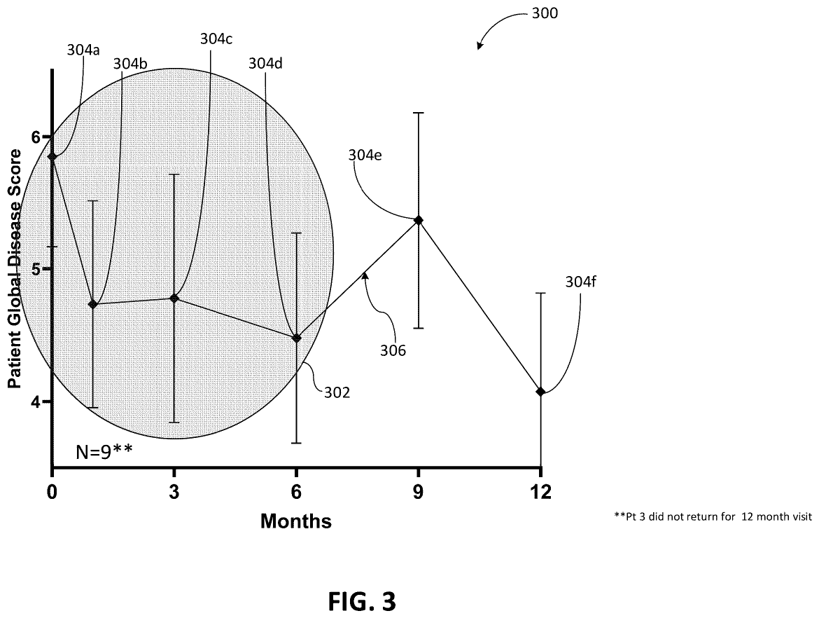

FIG. 3 at 300 provides an illustration the Patient Global Disease Score in participants with PM/DM who were administered ULSCs. Similar to the Physician Global Disease Score, there is a noted improvement from baseline to 6 months following administration. The Patient Global Disease Score is a patient reported outcome commonly used in autoimmune diseases. It is scored from 1-10, with 10 being the worse disease activity. The scoring is based on a patient assessment of themselves based on 42 scenarios. Elements 304a through 304f illustrate the varying score over a 12-month period.

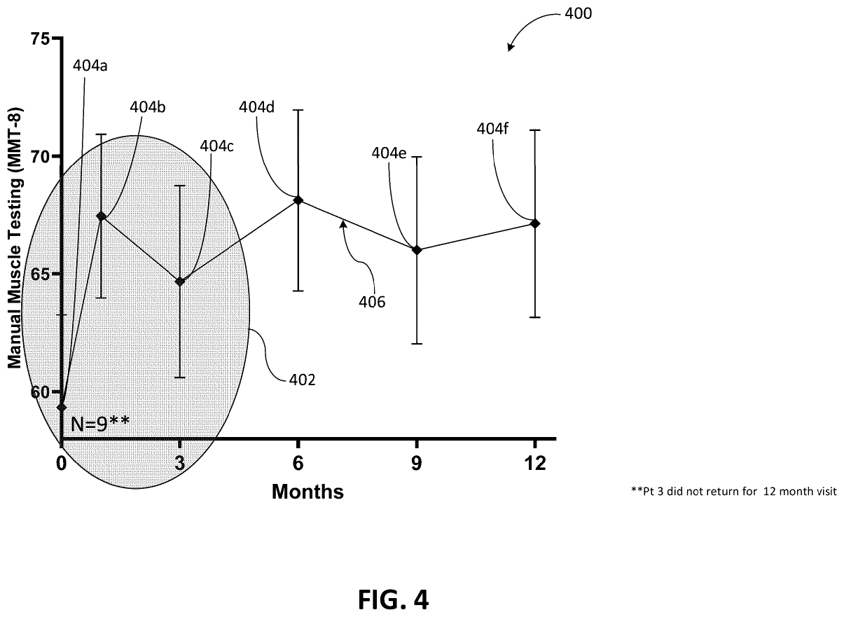

FIG. 4 at 400 provides an illustration with the average manual muscle testing (MMT-8) improvement for all participants who were administered ULSCs and maintained treatment from 1 month to 12 months. The improvement is compared to the baseline score prior to administration of ULSCs. The manual muscle testing (MMT-8) measure strength by the manual testing of 8 muscle groups. Strength is ranked from 0-80 with 80 being the highest muscle strength score. Elements 404a through 404f illustrate the varying score over a 12-month period.

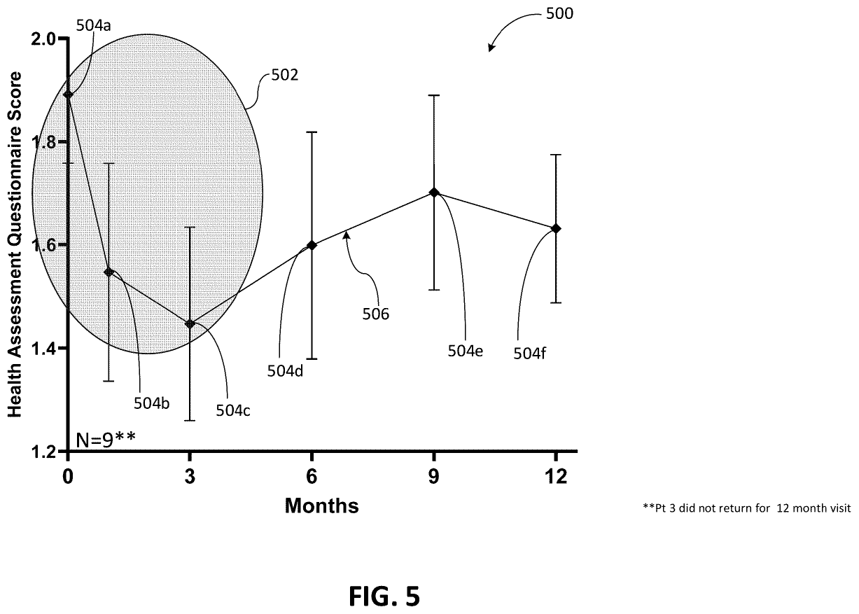

FIG. 5 at 500 provides an illustration of patient health following administration of ULSCs using the Health Assessment Questionnaire Disease Index (HAG-DI). The HAQ-DI is a patient reported outcome score that is ranked from 0-3, where 3 means completely disable and 0 means no disability. Elements 504a-504c exemplify a significant improvement in the HAQ from baseline to 3 months post-administration of ULSCs. Elements 504a through 504f display a general lowering of the HAQ-DI over 12 months as compared to baseline.

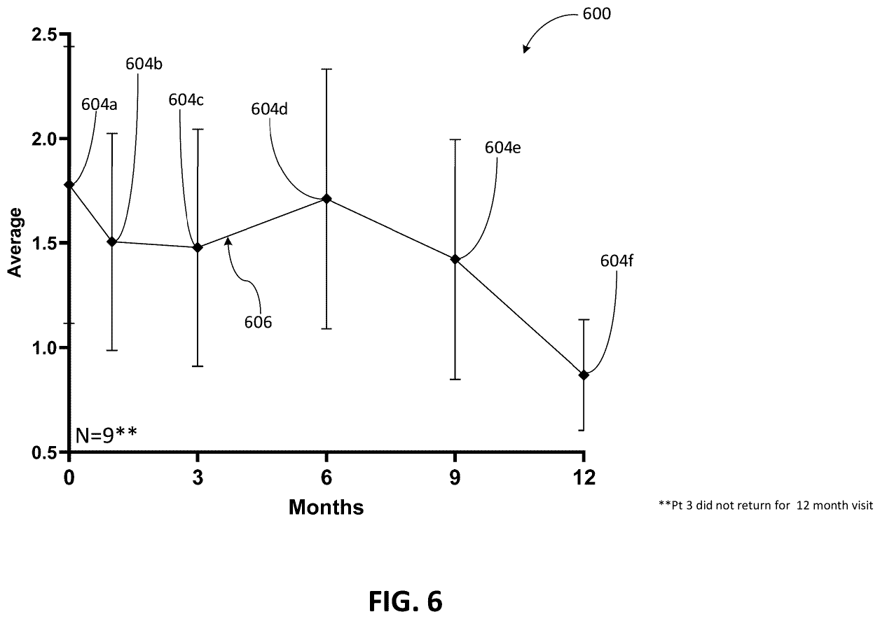

Referring to FIG. 6 at 600 provides an illustration representing the assessment of the average extra-muscular disease activity in all participants. The scoring system is ranked from 1-10 with higher scores representing more extra-muscular disease activity based on clinically defined assessments. Elements 604a-604f shows a significant lowering in extra-muscular disease activity score from baseline to 12 months post-administration of ULSCs which indicates less muscle disease. Elements 604a through 604f illustrate the varying score over a 12-month period.

FIG. 7 at 700 provides a graph displaying the total improvement score (TIS) of all participants over the course of a year. Elements 704a-704b show a significant increase one month after administration of ULSCs compared to the baseline score. This increase is maintained for the full length of the study. The TIS consists of a composite outcome from six core set measurements where the results are computed as a change between timepoints—where the improvement compared to a given initial timepoint is described as a percentage. The conventional agreement is that a minimally significant TIS is ≥20%. Between baseline and one-month post-administration of ULSCs, 6 of the 9 participants manifested clinically significant improvement in disease based on the TIS score. These improvements were observed in the context of reduced prednisone dosage. Elements 704a through 704f illustrate the varying score over a 12-month period.

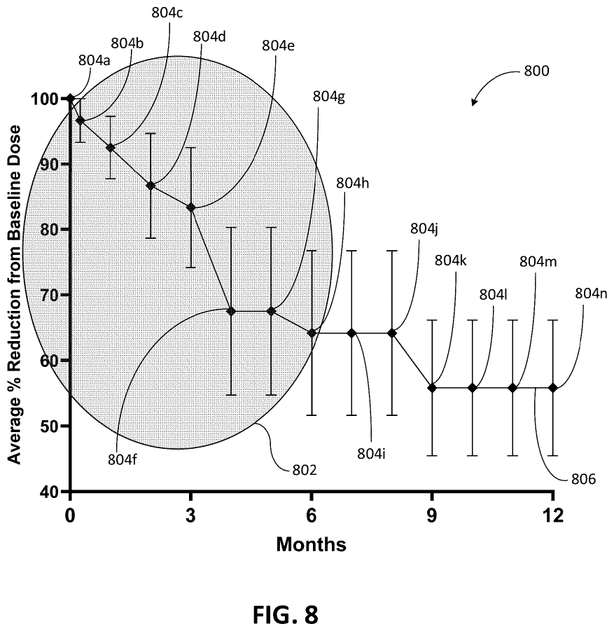

FIG. 8 at 800 provides a graph illustrating the average percent in reduction of prednisone between the baseline dose and up to 12 months post-administration of ULSCs. The average daily dose of prednisone was able to be tapered by more than 5.5 mg and the chronic use of as little as 2.6 mg daily lead to increased adverse side effects. Elements 804a through 804n illustrate the varying percentage reduction over a 12-month period.

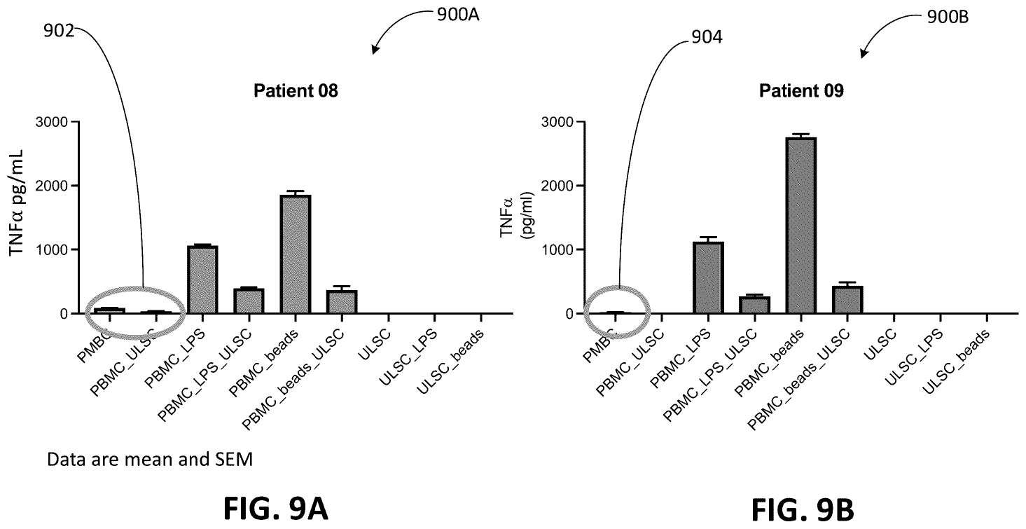

FIG. 9A at 900A provides an illustration of an in vitro assay mixing peripheral blood mononuclear cells (PBMC) from patient 08 with PM/DM with ULSCs to assess TNF-α. Before cell therapy, patients had a higher baseline release of TNF-α, but stimulated TNF-α release was repressed when PMBC was mixed with ULSCs. This pattern sustains even in groups where PBMC are stimulated with either lipopolysaccharide (LPS) or beads to secrete more TNF-α. TNF-α were assessed in conjunction with PBMC, PBMC plus ULSCs, PBMC stimulated with LPS, PBMC stimulated with beads and ULSCs, ULSCs alone, ULSCs stimulated with LPS, and ULSCs stimulated with beads.

FIG. 9B at 900B provides an illustration of an in vitro assay mixing peripheral blood mononuclear cells (PBMC) from patient 09 with PM/DM with ULSCs to assess TNF-α. Before cell therapy, patients had a higher baseline release of TNF-α, but stimulated TNF-α release was repressed when PMBC was mixed with ULSCs. This pattern sustains even in groups where PBMC are stimulated with either LPS or beads to secrete more TNF-α. TNF-α were assessed in conjunction with PBMC, PBMC plus ULSCs, PBMC stimulated with LPS, PBMC stimulated with beads and ULSCs, ULSCs alone, ULSCs stimulated with LPS, and ULSCs stimulated with beads.

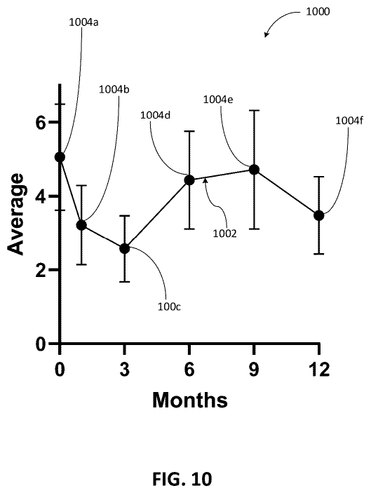

FIG. 10 at 1000 provides a graph illustrating the average reduction in pain for PM/DM participants from baseline scores to 12 months following administration of ULSCs. The pain scale is a patient reported outcome scale from 0-10 where 10 is the most pain and 0 is no reported pain. Elements 1004a through 1004f exemplify a general reduction in the amount of pain in PM/DM patients following administration of ULSCs. Elements 1004a through 1004c show that pain is reduced between 0 and 3 months before trending back up to baseline at 6 months.

### ULSC's Regulation of Specific Analytes in Patients With IIM

### IL-12 Levels in IIM Patients

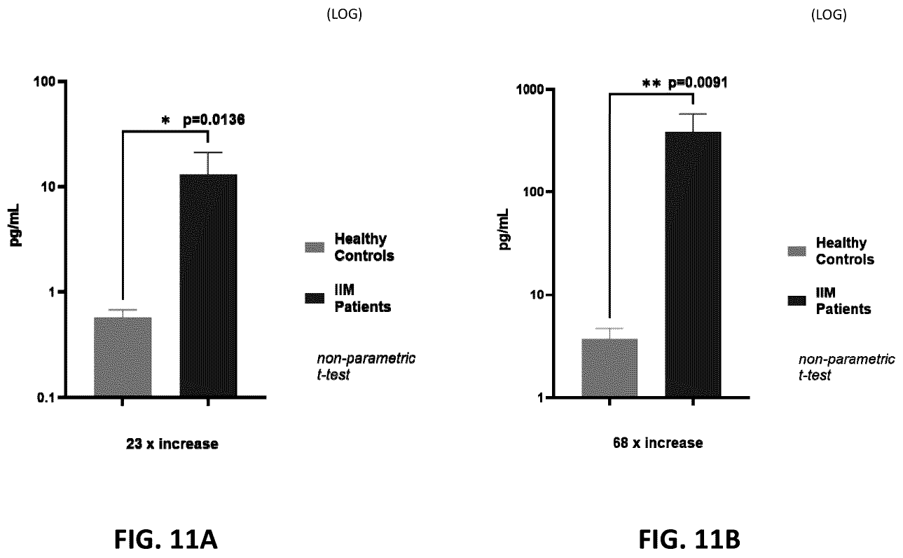

FIGS. 11A-11B provide graphs illustrating significant elevations of IL-12 in patients with idiopathic inflammatory myopathy (IIM) as compared to normal patients without IIM. FIGS. 11A and 11B show the results of IL-12 subunits IL-12p70 and IL-12p40, respectively, measured in patients with IIM and healthy patients. Patients with IIM showed increased amounts of IL-12p70 and IL-12p40 subunits compared to healthy controls. IL-12 is known to be proinflammatory, particularly in promotion of Th1 responses. IL-12p70 is involved in the development of autoimmune disease and plays a role in the pathogenesis of diseases such as psoriasis, and other such diseases. IL-12p70 also interlinks the innate and adaptive immune system via other cytokines such as, for example, IFN-gamma. IL-12p40 also pairs with p19 to form IL-23, another cytokine involved in inflammation and autoimmune diseases. IL-12p40 itself can act as a chemoattractant for macrophages and promote the migration of dendritic cells, both of which are involved in inflammatory responses.

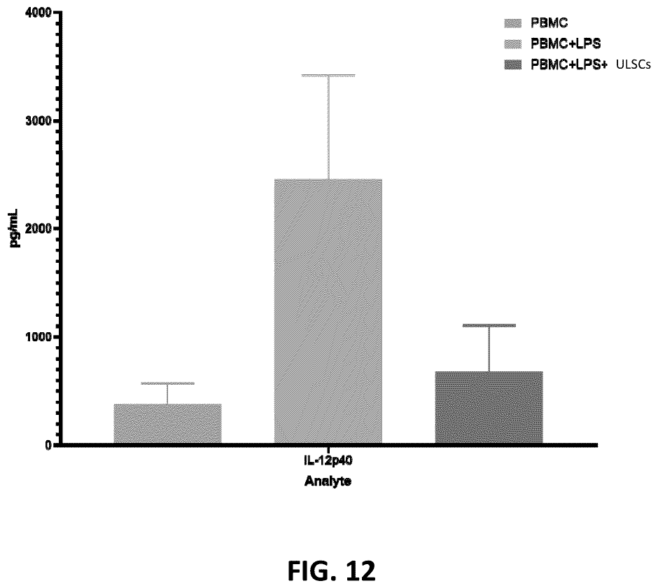

FIG. 12 provides a graph showing the average of IL-12p40 for IIM patients, showing that ULSCs reduce IL-12p40 in patients with IIM, and reduce the inflammatory response of patients' peripheral blood mononuclear cells (PBMCs) when stimulated with LPS. PBMCs alone from patients with IIM have an elevated amount of IL-12p40. When stimulated with LPS beadsP, IL-12p40 was shown to be significantly increased. When mixed with ULSCs, IL-12p40 was shown to be significantly reduced. These data suggest that ULSCs may significantly reduce the amount of IL-12p40 over 60%, and close to baseline.

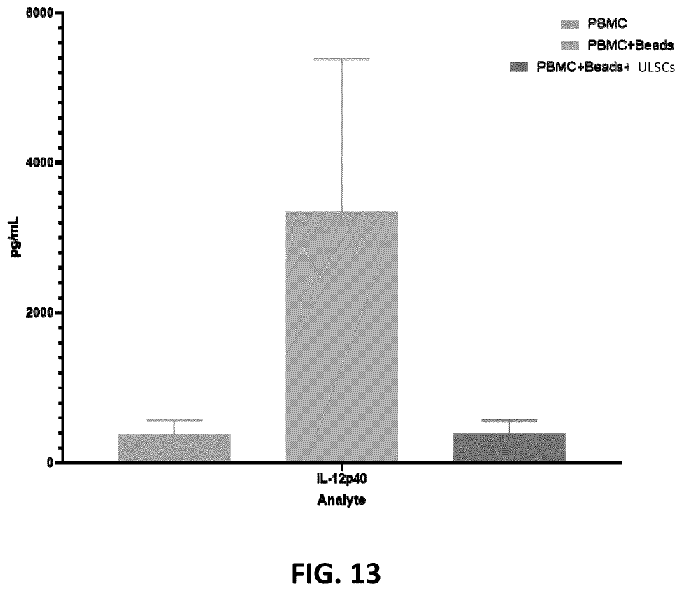

FIG. 13 provides a graph showing the average of IL-12p40 for IIM patients, showing that ULSCs reduce the IL-12p40 inflammatory response of patients' PBMCs when stimulated with inflammatory producing beads. PBMCs alone from patients with IIM have an elevated amount of IL-12p40. When stimulated with beads, IL-12p40 was shown to be significantly increased. When mixed with ULSCs, IL-12p40 was shown to be significantly reduced. Depending on the inflammatory stimulus, these data suggest that ULSCs may significantly reduce the amount of IL-12p40 by over 80% and close to baseline.

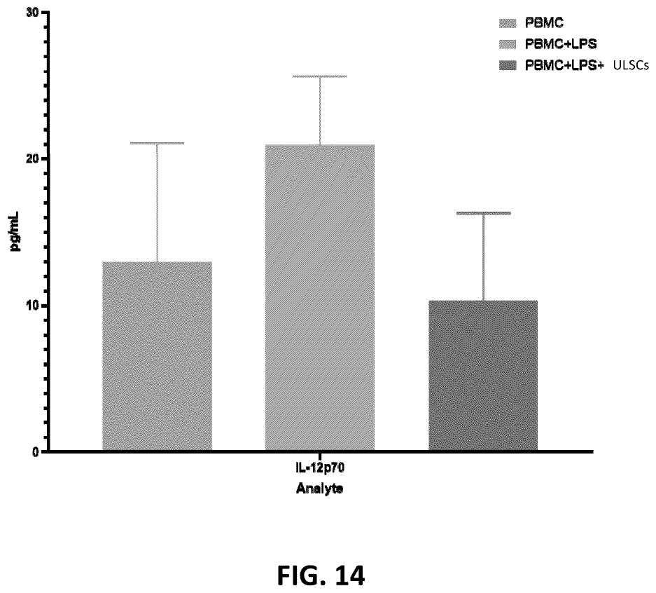

FIG. 14 provides a graph showing the average of IL-12p70 for IIM patients, showing that ULSCs reduce the IL-12p70 inflammatory response of patients' PBMCs when stimulated with LPS. PBMCs alone from patients with IIM have an elevated amount of IL-12p70. When stimulated with LPS beads, IL-12p70 is shown to be significantly increased. When adding stimulating beads and when the beads are mixed with ULSCs, IL-12p70 is shown to be reduced significantly to lower than their baseline. These data suggest that ULSCs may significantly reduce the amount of IL-12p70 by greater than 90% and below baseline, indicating a possible reduction in inflammation.

### MIP-1α Levels in IIM Patients

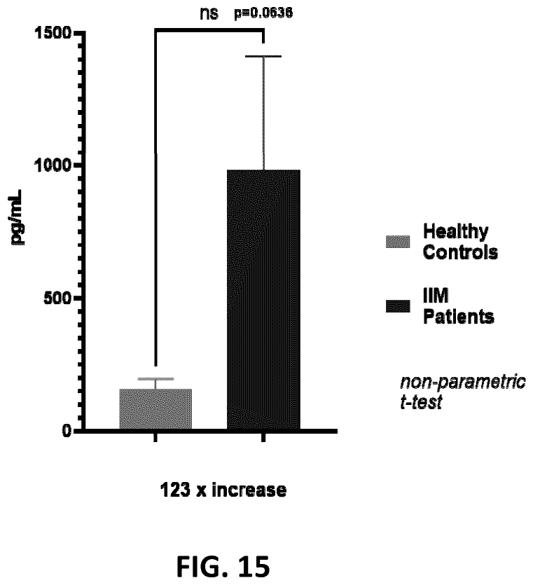

FIG. 15 provides a graph showing significant elevations of the cytokine macrophage inflammatory protein 1α (MIP-1α) in patients with IIM as compared to healthy controls. In IIM, amongst other autoimmune diseases, MIP-1α plays a role in attracting and activating immune cells such as monocytes, and subsets of lymphocytes to sites of inflammation.

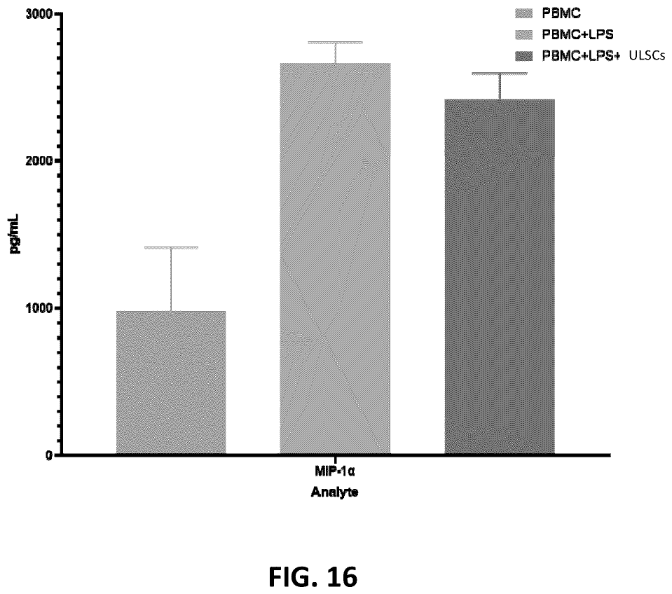

FIG. 16 provides a graph showing the average of MIP-1α for IIM patients, showing that ULSCs reduce the amount of MIP-1α in patients' PBMCs when stimulated with LPS. PBMCs alone from patients with IIM are shown to have an elevated amount of MIP-1α. When stimulated with LPS beads, MIP-1α is shown to be increased significantly. When mixed with ULSCs, MIP-1α is reduced. These data suggest that ULSCs may reduce the amount of MIP-1α which indicates a possible reduction in inflammatory response.

### IL-23 Levels in IIM Patients

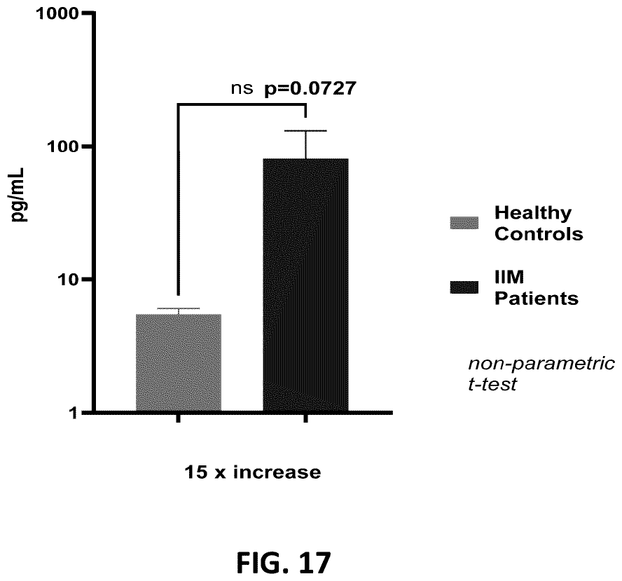

FIG. 17 provides a graph showing IL-23 levels for PBMC baseline, showing that patients with IIM have significantly higher levels of the cytokine IL-23 as compared to healthy controls. IL-23 plays a role in various autoimmune diseases such as inflammatory bowel disease, and also in IIM as suggested by these data. IL-23 promotes the differentiation and activation of TH-12 cells, which are immune cells known to contribute to inflammation in autoimmune conditions.

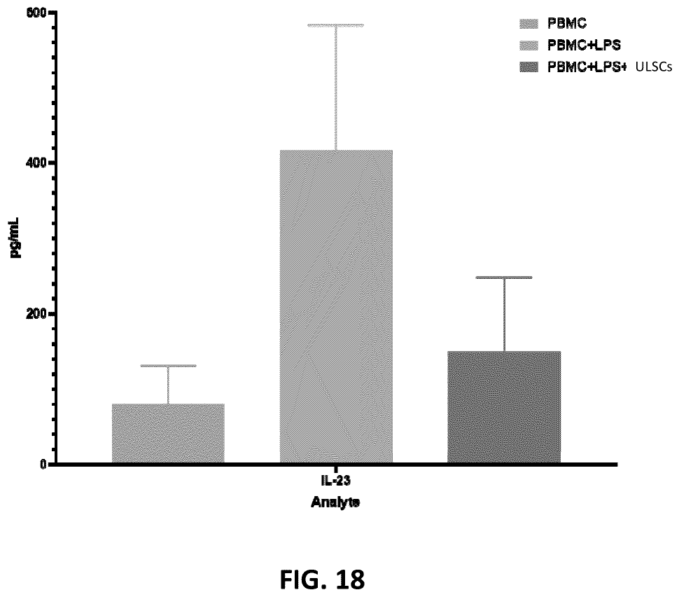

FIG. 18 provides a graph showing the average of IL-23 for IIM patients, showing that, in patients with IIM, PBMCs have significantly higher levels of IL-23, and when stimulated with LPS, IL-23 further increases significantly more. When IIM patients' PBMCs are stimulated with LPS and ULSCs are added, there is a significant reduction in IL-23. These data suggest that ULSCs reduce the inflammatory response mediated by IL-23.

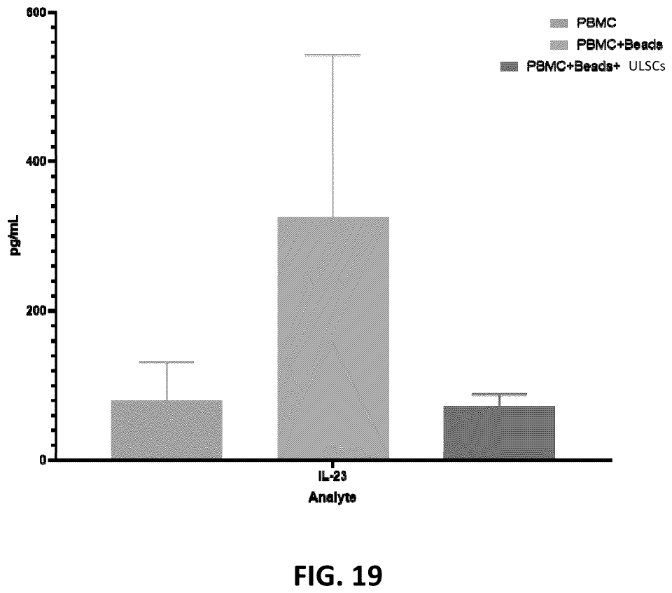

FIG. 19 provides a graph showing the average of IL-23 for IIM patients, showing that, in patients with IIM, PBMCs have significantly higher levels of IL-23, and when stimulated with immune stimulating beads, IL-23 further increases significantly more. When ULSCs are added to a patient's stimulated immune system, there is a significant decrease in the IL-23 mediated inflammatory response. These data suggest a reduction to below baseline inflammatory response in IIM patients.

1. A method of treating idiopathic inflammatory myopathy in an individual, comprising administering an effective amount of umbilical lining-derived stem cells to the individual in need thereof.

2. The method of claim 1, wherein the idiopathic inflammatory myopathy comprises dermatomyositis (DM) or polymyositis (PM).

3. The method of claim 1, wherein the effective amount of the umbilical lining-derived stem cells is at least about 50 million cells, about 100 million cells, or about 200 million cells.

4. (canceled)

5. (canceled)

6. The method of claim 1, wherein the administering comprises administering a single dose of the umbilical lining-derived stem cells.

7. The method of claim 1, wherein the administering comprises administering multiple doses of the umbilical lining-derived stem cells over a defined period of time.

8. A method of reducing steroid usage in an individual afflicted with idiopathic inflammatory myopathy, the method comprising administering an effective amount of umbilical lining-derived stem cells to the individual in need thereof.

9. The method of claim 8, wherein the idiopathic inflammatory myopathy comprises dermatomyositis (DM) or polymyositis (PM).

10. The method of claim 8, wherein the steroid comprises prednisone, prednisolone, methyl prednisone, dexamethasone, betamethasone, or triamcinolone, or derivates thereof.

11. The method of claim 8, wherein the effective amount of the umbilical lining-derived stem cells is at least about 50 million cells, about 100 million cells, or about 200 million cells.

12. (canceled)

13. (canceled)

14. The method of claim 8, wherein the administering comprises administering a single dose of the umbilical lining-derived stem cells.

15. The method of claim 8, wherein the administering comprises administering multiple doses of the umbilical lining-derived stem cells over a defined period of time.

16. (canceled)

17. (canceled)

18. (canceled)

19. (canceled)

20. (canceled)

21. (canceled)

22. (canceled)

23. (canceled)

24. A method of improving a symptom associated with idiopathic inflammatory myopathy in an individual, comprising administering an effective amount of umbilical lining-derived stem cells to the individual in need thereof.

25. The method of claim 24, wherein the idiopathic inflammatory myopathy comprises dermatomyositis (DM) or polymyositis (PM).

26. The method of claim 24, wherein the effective amount of the umbilical lining-derived stem cells is at least about 50 million cells, about 100 million cells, or about 200 million cells.

27. (canceled)

28. (canceled)

29. The method of claim 24, further comprising reducing or tapering a steroid dosage in the individual.

30. The method of claim 29, wherein the steroid comprises prednisone, prednisolone, methyl prednisone, dexamethasone, betamethasone, or triamcinolone, or derivates thereof.

31. The method of claim 24, wherein the administering comprises administering a single dose of the umbilical lining-derived stem cells.

32. The method of claim 24, wherein the administering comprises administering multiple doses of the umbilical lining-derived stem cells over a defined period of time.

33. The method of claim 24, wherein the symptom improvement is measured over at least a 1-month duration, at least a 2-month duration, or at least a 3-month duration.

34. (canceled)

35. (canceled)

36. The method of claim 24, wherein the symptom improvement correlates with a decrease in a cytokine level in the individual.

37. The method of claim 36, wherein the cytokine comprises TNF-α.

38. The method of claim 36, wherein the cytokine comprises IL-12.

39. The method of claim 36, wherein the cytokine comprises IL-12p40.

40. The method of claim 36, wherein the cytokine comprises IL-12p70.

41. The method of claim 36, wherein the cytokine comprises MIP-1α.

42. The method of claim 36, wherein the cytokine comprises IL-23.

43. An effective amount of umbilical lining-derived stem cells for use in treating an individual diagnosed with idiopathic inflammatory myopathy.

44. The umbilical lining-derived stem cells of claim 43, wherein the idiopathic inflammatory myopathy comprises dermatomyositis (DM) or polymyositis (PM).

45. The umbilical lining-derived stem cells of claim 43, wherein the effective amount comprises at least about 50 million cells, about 100 million cells, or about 200 million cells.

46. (canceled)

47. (canceled)
